(19) United States
(12) Patent Application Publication
(10) Pub. No.: US 2004/0023897 A1
(43) Pub. Date: Feb. 5, 2004

(54) Methods for preventing or treating disease mediated by toxin-secreting bacteria

(76) Inventor: Michael J. Caplan, Woodbridge, CT (US)

Correspondence Address: Choate, Hall & Stewart, Exchange Place, 53 State Street, Boston, MA, 02109, US

(21) Appl. No.: 10/292,392

(22) Filed: Nov. 12, 2002

Related U.S. Application Data
(60) Provisional application No. 60/337,548, filed on Nov. 13, 2001.
(60) Provisional application No. 60/338,618, filed on Nov. 13, 2001.

(51) Int. Cl. A61K 31/7048; A61K 31/65; A61K 31/43; A61K 31/545; A61K 31/496; A61K 31/47

(52) U.S. Cl. 514/29; 514/192; 514/200; 514/313; 514/153; 514/253.08

(57) ABSTRACT

Methods and pharmaceutical compositions for preventing and treating disease mediated by toxin-secreting bacteria. Inventive methods and compositions are suited to preventing or treating infections caused by bacterial toxins that enter host cells via receptor-mediated endocytosis (e.g., the anthrax and diphtheria toxins). Methods comprise a step of administering to an individual a pharmaceutical composition that includes an effective amount of an inhibitor of endosomal acidification. The inhibitor may be a primary amine, a carboxylic ionophore, or a selective inhibitor of the vacuolar proton pump (V-ATPase). The inhibitors of endosomal acidification may be employed in combination with other therapeutics such as antibiotics and antitoxins in order to prevent, treat or cure the disease.

The present invention describes techniques and reagents useful in the treatment of microbial infections, and particularly of infections with anthrax.

### RELATED APPLICATIONS

The present application claims priority to provisional applications U.S. Serial No. 60/337,548 and 60/338,618 both filed Nov. 13, 2001 which are incorporated herein by reference in their entirety.

### BACKGROUND OF THE INVENTION

Aggressive vaccination policies, combined with judicious administration of antibiotics, have reduced or eliminated the threats posed by many infectious diseases in the developed world. Unfortunately, however, there remain a variety of organisms whose infections cannot be effectively treated with current strategies. In some instances, vaccines have proven ineffective or have had unacceptable side effects; in others, the organism's cycle of infection provides few opportunities for available antibiotics to act.

Anthrax is one example of an infection that is poorly treated with existing therapies. An anthrax vaccine has been developed, but has not been demonstrated to be effective and is widely reputed to have serious negative side effects. The United States Centers for Disease Control (CDC) do not recommend use of the vaccine except for individuals who are at very high risk of being exposed to Bacillus anthracis, the bacterium that causes anthrax.

Anthrax infections can be treated with available antibiotics (e.g., penicillin G), but only if antibiotic therapy is initiated promptly after exposure. Bacillus anthracis, like many bacteria, secretes a toxin that poisons the individuals it infects. Once significant levels of toxin have accumulated, antibiotic therapy cannot help the infected individual because, even if all living bacteria are destroyed, the toxin will continue its damaging effects. Unfortunately, infected individuals often do not display symptoms of their infection until it is too late for antibiotics to be effective.

Accordingly, there remains a need for the development of improved treatments for infectious agents whose infections, like Bacillus anthracis, are poorly controlled by available vaccination and antibiotic therapies. In particular there is a need for therapeutic compounds that can be used as adjuncts to antibiotic therapy (e.g., compounds that inhibit the actions of the secreted toxin). Given that various governments and other organizations have apparently developed formulation and delivery systems for anthrax that allow it to be used as a biological weapon, and one that can be administered to individuals without notice so that timely antibiotic administration may not be possible, there is a particular need for the development of improved anthrax treatments.

### SUMMARY OF THE INVENTION

The present invention provides methods and pharmaceutical compositions for preventing and treating disease mediated by toxin-secreting bacteria. In particular, the prophylactic and therapeutic methods of the present invention are suited to preventing or treating infections caused by bacterial toxins that enter host cells via receptor-mediated endocytosis (e.g., the anthrax and diphtheria toxins). The present invention encompasses the recognition that inhibiting endosomal acidification may prevent such toxins from entering host cells. The inventive methods comprise a step of administering to an individual a pharmaceutical composition that includes an effective amount of an inhibitor of endosomal acidification. In one aspect, the inhibitor of endosomal acidification is a primary amine such as methylamine, ammonium chloride, chloroquine, amantadine, rimantadine, and dansylcadaverine. In another aspect, the inhibitor of endosomal acidification is a carboxylic ionophore such as monensin and nigerin. In another aspect, the inhibitor of endosomal acidification is a selective inhibitor of the vacuolar proton pump (V-ATPase) such as bafilomycin A₁, concanamycin A, salicylihalamide A, lobatamides A-F, and oximidines I and II. Typically, the inhibitors of endosomal acidification will be employed in combination with other therapeutics such as antibiotics and antitoxins in order to prevent, treat or cure the disease. In many embodiments of the invention, inventive compositions will be administered after symptoms of infection have become manifest.

### DESCRIPTION OF CERTAIN PREFERRED EMBODIMENTS

The present application mentions various patents, scientific articles, and other publications. The contents of each such item are hereby incorporated by reference. In addition, the contents (as of the filing date of the application) of all websites referred to herein are incorporated by reference.

Introduction

Many bacterial toxins, notably those that act within the cytosol of host cells, consist of two components: one component (subunit A) is responsible for the enzymatic activity of the toxin; the other component (subunit B) is concerned with binding to a specific receptor on the host cell membrane and transferring the enzyme across the membrane. The enzymatic component is not active until it is released into the host cell cytosol. Isolated A subunits are enzymatically active but lack binding and cell entry capability. Isolated B subunits may bind to target cells, but they are non-toxic.

There are a variety of ways that toxin subunits may be synthesized and arranged: A-B or A-7B indicates subunits synthesized separately and associated by non-covalent bonds; A/B denotes subunit domains of a single protein that may be separated by proteolytic cleavage; A+B indicates separate protein subunits that interact at the host cell surface; 7B indicates that the binding domain is a heptamer of identical B subunits.

There are at least two mechanisms by which bacterial toxins may enter the cytosol of host cells. In one mechanism, subunit B of the toxin binds to a specific receptor on the target cell and induces the formation of a pore in the membrane through which subunit A is translocated to the host cell cytosol. In an alternative mechanism (e.g., that followed by the anthrax and diphtheria toxins), the B subunit of the toxin binds to a specific receptor on the host cell, subunit A binds to the B subunit to form a membrane-bound toxin complex, and the complex is taken into the cell by receptor-mediated endocytosis. The toxin complex is thereby internalized within the host cell in a membrane-enclosed vesicle called an endosome. H⁺ ions subsequently enter the endosome via ATP driven proton pumps (V-ATPases) thereby lowering the lumenal pH. Endosomal acidification somehow causes the A and B subunits to separate and the A subunit to be translocated to the host cell cytosol. The B subunit remains in the endosome and is recycled to the host cell surface.

Anthrax toxin consists of three antigenically distinct subunits: a single receptor-binding subunit (protective antigen, PA) which, when activated by proteloytic cleavage, self-assembles to form a ring shaped heptamer on the surface of host cells, and two enzymatic subunits (edema factor, EF and lethal factor, LF) that competitively bind with the heptameric structure. Once the resulting membrane-associated toxin complex has been endocytosed and the endosomal compartment has been acidified, the heptameric structure inserts into the endosomal membrane and mediates translocation of EF or LF into the cytosol of the host cell. The combination of PA and EF has been observed to cause edema in animals in experiments, and the combination of PA and LF causes death. EF is a calmodulin-dependent adenylate cyclase that has an inhibitory effect on professional phagocytes, and LF is a zinc protease that acts specifically on macrophages, causing their death and the death of the host (see, Leppla, “Anthrax Toxins” in Bacterial toxins and virulence factors in diseases. Handbook of Natural Toxins, Vol. 8, pp. 543-572, Ed. by Moss et al., Dekker, New York, N.Y., 1995; Miller et al., Biochemistry 38:10432, 1999; and Duesbury et al., Science 280:734, 1998).

Diphtheria toxin is derived from a single “tox” protein. Two processing steps are necessary before the “tox” protein can be secreted from Corynebacterium diphtheriae (the bacterium that causes diphtheria), namely proteolytic cleavage of a leader sequence and subsequent cleavage of the product into two subunits (A and B) that remain attached by a disulfide bond. The diphtheria toxin includes three functional regions: a receptor-binding region and a translocation region on the B subunit and an enzymatic region on the A subunit. The receptor binding region of the B subunit binds to the heparin-binding epidermal growth factor receptor, which is present on the surface of many eukaryotic cells, particularly heart and nerve cells. After the toxin becomes attached to the host cell, it is engulfed in an endocytic vescicle. The A subunit is released into the host cell cytosol, an event mediated by the B subunit translocation region and triggered by endosomal acidification. Once within the cytosol, the A subunit inhibits host cell protein synthesis by catalyzing adenosine diphosphate-ribosylation of elongation factor 2 (EF-2), a factor required for the movement of nascent peptide chains on ribosomes.

The present invention encompasses the recognition that the translocation of enzymatic subunits of certain bacterial toxins (i.e., those that enter host cells via receptor-mediated endocytosis such as the anthrax and diphtheria toxins) can be prevented by inhibiting endosomal acidification.

Compounds

Compounds that can be used as inhibitors of endosomal acidification for the present invention include primary amines such as methylamine, ammonium chloride, chloroquine, amantadine, rimantadine, dansylcadaverine, and derivatives thereof. The phrase, “inhibitors of endosomal acidification”, as used herein, denotes any compound that causes an increase in pH within the lumen of endosomes. Preferably the pH in the presence of an inhibitor of endosomal acidification is sufficiently greater than in the absence of the inhibitor to prevent translocation of enzymatic subunits of bacterial toxins. In unprotonated form, primary amines can pass through the endosomal membrane. Once inside endosomes, the weakly basic primary amines become protonated, thereby buffering the lumenal environment of the endosome and preventing acidification. Once protonated, the primary amines cannot cross the endosomal membrane and therefore remain trapped within endosomes. Vascular swelling may occur because of osmotic imbalance.

Compounds that can be used as inhibitors of endosomal acidification for the present invention also include carboxylic ionophores such as monensin, nigerin, and derivatives thereof. Carboxylic ionophores have a linear structure with a carboxyl group on one end and one or two hydroxyls on the other. They cyclize by head-to-tail hydrogen bonding and cross membranes with the carboxyl group either protonated or complexed to an ion. Nigericin, for example, will cross the endosomal membrane carrying either H⁺ or K⁺. It therefore functions as a H⁺/K⁺ exchanger, passively countering the active build of a proton gradient and hence preventing endosomal acidification. Because carboxylic ionophores do not carry a net charge across the membrane, transport is not affected by the membrane potential nor does it contribute to the creation of a membrane potential.

Compounds that can be used as inhibitors of endosomal acidification for the present invention further include selective inhibitors of the vacuolar proton pump (V-ATPase) such as N-ethylmaleimide, N,N′-dicyclohexylcarbodiimide, bafilomycin A₁, concanamycin A, mycotoxin destruxin B, salicylihalamide A, lobatamides A-F, oximidines I and II, and derivatives thereof. V-ATPases generate and maintain an acidic environment within the lumen of early endosomes, late endosomes, and lysosomes (see, Arai et al., Biochemistry 26:6632, 1987; Arai et al., J. Biol. Chem. 268:5649, 1993; and Moriyama and Nelson, J. Biol. Chem. 264:18445, 1989, for reviews, see, Stevens and Forgac, Annu. Rev. Cell. Dev. Biol. 13:779, 1997 and Finbow and Harrison, Biochem. J. 324:697, 1997).

V-ATPases are electrogenic, i.e., they create an electric potential difference across the membrane. Continued proton transport requires dissipation of the membrane potential, which occurs primarily through the action of a chloride channel (see, Glickman et al., J. Cell Biol. 97:1303, 1983 and Arai et al., Biochemistry 28:3075, 1989). V-ATPases are structurally and pharmacologically distinct from plasma membrane (P-) and mitochondrial (F-) proton ATPases. According to the current view, V-ATPases are multi-subunit enzymes composed of two functional domains: a transmembraneous proton channel (V₀ domain) and a peripheral catalytic domain (V₁ domain) (for reviews, see, Stevens and Forgac, Annu. Rev. Cell. Dev. Biol. 13:779, 1997 and Finbow and Harrison, Biochem. J. 324:697, 1997). The highly hydrophopic V₀ domain consists of six copies of 17 kDa subunits (c) that form the proton channel and a single copy of 100 kDa (a), 38 kDa (d), 19 kDa (c″) and 17 kDa (c′) subunits. The catalytic domain V₁ is responsible for ATP hydrolysis and consists of three copies of 70 kDa (A) and 60 kDa (B) subunits and a single copy of 40 kDa (C), 34 kDa (D), 33 kDa (E), 14 kDa (F), 13 kDa (G), and 50-57 kDa (H) subunits that form the proton channel.

A variety of compounds have been found to interact with the V-ATPases and cause inhibition of both ATP hydrolysis and proton translocation activities (see, Finbow and Harrison, Biochem. J. 324:697, 1997). Although any of these compounds may be used for the present invention, compounds that specifically inhibit V-ATPases are preferred. V-ATPases are acutely sensitive to N-ethylmaleimide (NEM), with inhibition of ATP hydrolysis occurring at low micromolar concentrations (see, Xie and Stone, J. Biol. Chem. 263:9859, 1988; Cidon and Nelson, J. Biol. Chem. 261:9222, 1986; Moriyama and Nelson, J. Biol. Chem. 262:14723, 1987; Bowman et al., Proc. Natl. Acad. Sci. USA 83:48, 1986; Percy et al., Biochem. J. 231:557, 1985; and Young et al., Proc. Natl. Acad. Sci. USA 85:9590, 1988). Inhibition arises through modification of a cysteine residue in the conserved P-loop sequence of subunit A (see, Taiz et al., Biochim. Biophys. Acta 1194:329, 1994 and Feng and Forgac, J. Biol. Chem. 267:5817, 1992).

The sensitivity of the V-ATPases to N,N′-dicyclohexylcarbodiimide (DCCD) has been widely documented (see, Uchida et al., J. Biol. Chem. 260:1090, 1985; Mandala and Taiz, J. Biol. Chem. 261:12850, 1986; Sun et al., J. Biol. Chem. 262:14790, 1987; Bowman, J. Biol. Chem. 258:15238, 1983; and Kakinuma et al., J. Biol. Chem. 256:10859, 1981), and the effect of this inhibitor has been shown to be exerted through covalent modification of the 17 kDa subunit c (see, Arai et al., J. Biol. Chem. 262:11006, 1987; Kaestner et al., J. Biol. Chem. 263:1282, 1988; and Rea et al., J. Biol. Chem. 262:14745, 1987). More specifically, DCCD reacts with the side chain of a conserved acidic residue present in a transmembrane segment of the 17 kDa subunit c to yield a stable dicyclohexyl-O-acylisourea moiety (see, Nalecz et al., Methods Enzymol. 20:86, 1986; and Hassinen and Vuokila, Biochim. Biophys. Acta 1144:107, 1993). Micromolar concentrations of DCCD are sufficient to give complete and irreversible inhibition of proton pumping, although only 60 to 80% of ATP hydrolyzing activity may be lost. Modification of a single site per enzyme is sufficient to abolish proton translocation entirely (see, Arai et al., J. Biol. Chem. 262:11006, 1987), indicating some form of allostery or co-operativity in subunit c (see, Pali et al., Biochemistry 34:9211, 1995).

Bafilomycin A₁, and the related compound concanamycin A, are macrolide antibiotics consisting of a 16- or 18-membered lactone ring which are highly specific inhibitors of the V-ATPases (see, Bowman et al., Proc. Natl. Acad. Sci. USA, 85:7972, 1988; Drose et al., Biochemistry 32:3902, 1993). Reversible inhibition of proton translocation occurs at sub-micromolar concentrations (see, Kane et al., J. Biol. Chem. 264:19236, 1989; Bowman et al., Proc. Natl. Acad. Sci. USA., 85:7972, 1988; Hanada et al., Biochem. Biophys. Res. Commun. 170:873, 1990; and Crider et al., J. Biol. Chem. 269:17379, 1994), and involves interaction with at least one protein component of the V₀ domain (see, Zhang et al., J. Biol. Chem. 269:23518, 1994; Crider et al., J. Biol. Chem. 269:17379, 1994; and Rautiala et al., Biochem. Biophys. Res. Commun. 194:50, 1993).

The cyclic peptide mycotoxin destruxin B (see, Muroi et al., Biochem. Biophys. Res. Commun. 205:1358, 1994) and the benzolactone enamides salicylihalamide A, lobatamides A-F, and oximidines I and II (see, Boyd et al., J. Pharmacol. Exp. Ther. 297:114, 2001) have also been demonstrated to be highly specific and efficacious inhibitors of V-ATPases.

It will be appreciated that certain chemical derivatives of the compounds set forth above may also be used as inhibitors of endosomal acidification for the present invention. The phrase, “chemical derivative”, as used herein, denotes any chemical derivative which, upon administration to an individual, inhibits endosomal acidification in substantially the same way as a compound as otherwise described herein. It is to be understood that the present invention encompasses the use of chemical derivatives identified using direct and indirect high throughput screening methods. For example, direct screens may use in vitro fluorescence imaging techniques to measure changes in endosomal pH (see, for example, Plant et al., J. Biol. Chem. 274:37270, 1999; van Weert et al., J. Cell. Biol. 130:821, 1995; Gurich et al., J. Clin. Invest. 87:1547, 1991; and Yamashiro and Maxfield, J. Cell. Biol. 105:2723, 1987); while indirect screens may monitor the translocation of the toxin enzymatic subunits to the host cell cytosol and/or monitor the catalytic effects of enzymatic subunits within the cytosol (see, for example, Wesche et al., Biochemistry 37:15737, 1998; Blaustein et al, Proc. Natl. Acad. Sci. USA 86:2209, 1989; and Gordon et al., Infect. Immun. 56:1066, 1988).

Chemical derivatives include compounds, as described herein, that have been substituted with any number of substituents or functional moieties. In general, the term “substituted” refers to the replacement of hydrogen radicals in a given structure with the radical of a specified substituent. When more than one position in any given structure is substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. For purposes of this invention, heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in the treatment of disease mediated by toxin-secreting bacteria. The term “stable”, as used herein, preferably refers to compounds which possess stability sufficient to allow manufacture and which maintain the integrity of the compound for a sufficient period of time to be detected and preferably for a sufficient period of time to be useful for the purposes detailed herein.

It will also be appreciated that pharmaceutically acceptable derivatives of the compounds set forth above may also be used as inhibitors of endosomal acidification for the present invention. The phrase, “pharmaceutically acceptable derivative”, as used herein, denotes any pharmaceutically acceptable salt, ester, or salt of such ester, of such compound, or any other adduct or derivative which, upon administration to a patient, is capable of providing (directly or indirectly) a compound as otherwise described herein, or a metabolite or residue thereof. Pharmaceutically acceptable derivatives thus include among others pharmaceutically acceptable pro-drugs.

As used herein, the term “pharmaceutically acceptable salt” refers to those salts which are, within the scope of sound medical judgment, suitable for use in contact with the tissues of human and non-human animals without undue toxicity, irritation, allergic response and the like, and are commensurate with a reasonable benefit/risk ratio. Pharmaceutically acceptable salts are well known in the art. For example, Berge et al. describe pharmaceutically acceptable salts in detail (see, Berge et al., J. Pharm. Sci. 66:1, 1977). The salts can be prepared in situ during the final isolation and purification of the compounds of the invention, or separately by reacting the free base function with a suitable organic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hernisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Additionally, as used herein, the term “pharmaceutically acceptable ester” refers to esters which hydrolyze in vivo and include those that break down readily in the body of an individual to leave the parent compound or a salt thereof. Suitable ester groups include, for example, those derived from pharmaceutically acceptable aliphatic carboxylic acids, particularly alkanoic, alkenoic, cycloalkanoic, and alkanedioic acids. Examples of particular esters includes formates, acetates, propionates, butyrates, acrylates and ethylsuccinates.

Furthermore, the term “pharmaceutically acceptable pro-drugs” as used herein refers to those pro-drugs of the compounds of the present invention which are, within the scope of sound medical judgment, suitable for use in contact with the tissues of humans and non-human animals with undue toxicity, irritation, allergic response, and the like, commensurate with a reasonable benefit/risk ratio, and effective for their intended use, as well as the zwitterionic forms, where possible, of the compounds of the invention. The term “pro-drug” refers to derivatives of a compound, usually with significantly reduced pharmacological activity, which contains an additional moiety which is susceptible to removal in vivo yielding the parent molecule as the pharmacologically active species. An example of a pro-drug is an ester which is cleaved in vivo to yield a compound of interest. Pro-drugs of a variety of compounds, and materials and methods for derivatizing the parent compounds to create the pro-drugs, are known and may be adapted to the present invention. A thorough discussion of pro-drugs is provided in Higuchi and Stella, Pro-drugs as Novel Delivery Systems, Vol. 14 of the A.C.S. Symposium Series, and in Bioreversible Carriers in Drug Design, Ed. by Roche, American Pharmaceutical Association and Pergamon Press, 1987.

Pharmaceutical Compositions

This invention also provides a pharmaceutical composition for the treatment and prophylaxis of diseases or undesirable conditions which are mediated by bacterial toxins, the pharmaceutical composition comprising at least one of the foregoing compounds, a chemical derivative thereof, or a pharmaceutically acceptable derivative thereof, as inhibitors of endosomal acidification, and at least one pharmaceutically acceptable excipient or additive. Preferably the bacterial toxins enter host cells via receptor-mediated endocytosis, e.g., the toxins secreted by Bacillus anthracis and Corynebacterium diphtheriae. Preferably the excipient or additive is pharmaceutically innocuous.

In certain preferred embodiments, the inventive pharmaceutical compositions optionally further comprise one or more additional therapeutic agents. In certain embodiments, the additional therapeutic agent is an antibiotic for the treatment of bacterial infection, as discussed in more detail herein.

For example, when treating infection by Bacillus anthracis, the pharmaceutical composition may further include an effective amount of penicillin G, erythromycin, doxycycline, chloramphenicol, ciprofloxacin, cephalexin, cefazolin, and/or cefadroxil. Penicillin G, and the first generation cephalosporins cephalexin, cefazolin, and cefadroxil are beta-lactam antibiotics that inhibit bacterial cell wall synthesis by interacting with bacterial enzymes that cross-link peptidoclycans. The macrolide erythromycin is thought to inhibit protein synthesis by blocking translocation within the 50S ribosomal subunit. Chloramphenicol is thought to inhibit protein synthesis by preventing tRNAs from binding to mRNA codons and by blocking peptide bond formation within the 50S ribosomal subunit. The tetracycline doxycycline is thought to inhibit protein synthesis by binding to the smaller 30S ribosomal subunit and thereby preventing tRNAs from binding to mRNA codons. Finally, the first generation fluoroquinolone ciprofloxacin is thought to inhibit bacterial chromosome replication by binding to DNA gyrase. For further details on common antibiotic treatments for anthrax, see, Hart and Beeching, British Medical J. 323:1017, 2001 and Dixon et al., N. Engl. J. Med. 341:815, 1999.

Similarly, when treating infection by Corynebacterium diphtheriae, the pharmaceutical composition may further include an effective amount of erythromycin, clindamycin, rifampin, cephalexin, cefazolin, and/or cefadroxil. Clindamycin is thought to inhibit protein synthesis by binding to the 50S ribosomal subunit and blocking translocation. Rifampin is also thought to inhibit protein synthesis, but via inhibition of DNA-dependent RNA polymerase at the initiation step. For further details on common antibiotic treatments for diphtheria, see, Bisgsard et al., Am. J. Public Health 88:787, 1988; CDC Morb. Mortal. Wkly. Rep. 46:502, 1997; Dittmann, Internat. Assoc. Biol. Standard 25:179, 1997; and Farizo et al., Clin. Infect. Dis. 16:59, 1993.

As described above, the pharmaceutical compositions of the present invention additionally comprise a pharmaceutically acceptable carrier, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Fifteenth Edition, E. W. Martin (Mack Publishing Co., Easton, Pa., 1975) discloses various carriers used in formulating pharmaceutical compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutical composition, its use is contemplated to be within the scope of this invention.

Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Uses of Compounds of the Invention

The invention further provides a method for the treatment and prophylaxis of diseases or undesirable conditions which are mediated by bacterial toxins, more specifically bacterial toxins that enter host cells via receptor-mediated endocytosis. The method involves administering a therapeutically effective amount of one of the foregoing compounds, a chemical derivative thereof, or a pharmaceutically acceptable derivative thereof to an individual in need of it.

The term “individual”, as used herein, refers to humans as well as non-human animals, including, for example, mammals, birds, reptiles, amphibians, and fish. Preferably, the non-human animal is a mammal (e.g., a rodent, a mouse, a rat, a rabbit, a monkey, a dog, a cat, a primate, or a pig). An animal may be a transgenic animal. It will be appreciated that preferred individuals have been exposed to or are at a high risk of being exposed to a bacterial toxin that invades host cells via receptor-mediated endocytosis.

In certain embodiments of the present invention a “therapeutically effective amount” of the inventive compound or pharmaceutical composition is that amount effective for inhibiting endosomal acidification, or is an amount that is effective for inhibiting the translocation of the enzymatic subunit of a bacterial toxin, which translocation is believed to be involved in the bacterial infection, although the present invention is not intended to be bound by any particular theory.

The compounds and pharmaceutical compositions, according to the method of the present invention, may be administered using any amount and any route of administration effective for inhibiting endosomal acidification, or for treating bacterial infections. Thus, the expression “effective amount” as used herein, refers to a sufficient amount of agent to inhibit endosomal acidification, or to treat a bacterial infection. The exact amount required will vary from subject to subject, depending on the species, age, and general condition of the subject, the severity of the infection, the particular compound, its mode of administration, and the like. The compounds of the invention are preferably formulated in dosage unit form for ease of administration and uniformity of dosage. The expression “dosage unit form” as used herein refers to a physically discrete unit of compound appropriate for the individual to be treated. It will be understood, however, that the total daily usage of the compounds and pharmaceutical compositions of the present invention will be decided by the attending physician within the scope of sound medical judgment. The specific therapeutically effective dose level for any particular individual will depend upon a variety of factors including the disorder being treated and the severity of the disorder; the activity of the specific compound employed; the specific pharmaceutical composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

Furthermore, after formulation with an appropriate pharmaceutically acceptable carrier in a desired dosage, the pharmaceutical compositions of this invention can be administered to individuals orally, rectally, parenterally, intracisternally, intravaginally, intraperitoneally, topically (as by powders, ointments, or drops), bucally, as an oral or nasal spray, or the like, depending on the severity and location of the infection being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound, it is often desirable to slow the absorption of the compound from subcutaneous or intramuscular injection. This may be accomplished by the use of a liquid suspension of crystalline or amorphous material with poor water solubility. The rate of absorption of the drug then depends upon its rate of dissolution that, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissues.

Compositions for rectal or vaginal administration are preferably suppositories which can be prepared by mixing the compounds of this invention with suitable nonirritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the compound is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar—agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Combination Therapy

It will also be appreciated that the compounds and pharmaceutical compositions of the present invention can be employed in combination therapies, that is, the compounds and pharmaceutical compositions can be administered concurrently with, prior to, or subsequent to, one or more other desired therapeutics. The particular combination of therapeutics to employ in a combination regimen will take into account compatibility of the desired therapeutics and the desired therapeutic effect to be achieved. It will also be appreciated that the therapies employed may achieve a desired effect for the same disorder (for example, an inventive compound may be administered concurrently with a different inventive compound), or they may achieve different effects (e.g., control of any adverse effects). For example, other therapies or therapeutic agents that may be used in combination with the inventive compounds agents of the present invention include antibiotics, as discussed earlier. It is further intended that the inventive compounds of the present invention be used in combination with so called “antitoxins”, i.e., antibodies directed to the bacterial toxins as described in greater detail in U.S. Patent Serial No. 60/337,548, entitled “Anti-Toxins”, filed on Nov. 13, 2001, the entire contents of which are attached herewith as Exhibit A and incorporated herein by reference.

Treatment Kits

In other embodiments, the present invention relates to a kit for conveniently and effectively carrying out the methods in accordance with the present invention. In general, the pharmaceutical pack or kit comprises one or more containers filled with one or more of the ingredients of the pharmaceutical compositions of the invention. Such kits are especially suited for the delivery of solid oral forms such as tablets or capsules. Such a kit preferably includes a number of unit dosages, and may also include a card having the dosages oriented in the order of their intended use. If desired, a memory aid can be provided, for example in the form of numbers, letters, or other markings or with a calendar insert, designating the days in the treatment schedule in which the dosages can be administered. Alternatively, placebo dosages, or calcium dietary supplements, either in a form similar to or distinct from the substituted purine dosages, can be included to provide a kit in which a dosage is taken every day. Optionally associated with such container(s) can be a notice in the form prescribed by a governmental agency regulating the manufacture, use or sale of pharmaceutical products, which notice reflects approval by the agency of manufacture, use or sale for human administration.

### Other Embodiments

Other embodiments of the invention will be apparent to those skilled in the art from a consideration of the specification or practice of the invention disclosed herein. It is intended that the specification and examples be considered as exemplary only, with the true scope of the invention being indicated by the following claims.

### EXHIBIT A

Provisional Application U.S. Serial No.: 60/337,548 Filed: Nov. 13, 2001

### BACKGROUND OF THE INVENTION

Aggressive vaccination policies, combined with judicious administration of antibiotics, have reduced or eliminated the threats posed by many infectious diseases in the developed world. Unfortunately, however, there remain a variety of organisms whose infections cannot be effectively treated with current strategies. In some instances, vaccines have proven ineffective or have had unacceptable side effects; in others, the organism's cycle of infection provides few opportunities for available antibiotics to act.

Anthrax is one example of an infection that is poorly treated with existing therapies. An anthrax vaccine has been developed, but has not been demonstrated to be effective and is widely reputed to have serious negative side effects. The Centers for Disease Control in the United States do not recommend use of the vaccine except for individuals who are at very high risk of being exposed to Bacillus anthracis, the bacterium that causes anthrax.

Anthrax infections can be treated with available antibiotics, but only if antibiotic therapy is initiated promptly after exposure. Anthrax, like many bacteria, secretes a toxin that poisons the individuals it infects. Once significant levels of toxin have accumulated, antibiotic therapy cannot help the infected individual because, even if all living bacteria are destroyed, the toxin will continue its damaging effects. Unfortunately, infected individuals often do not display symptoms of their infection until it is too late for antibiotics to be effective.

There remains a need for the development of improved treatments for infectious agents whose infections, like anthrax, are poorly controlled by available vaccination and antibiotic therapies. There is a particular need for the development of improved treatments for infections of toxin-producing bacteria such as Bacillus anthracis (anthrax). Given that various governments and other organizations have apparently developed formulation and delivery systems for anthrax that allow it to be used as a biological weapon, and one that can be administered to individuals without notice so that timely antibiotic administration may not be possible, there is an immediate need for the development of improved anthrax treatments.

### SUMMARY OF THE INVENTION

The present invention provides anti-toxin compositions for use in treating infections of toxin-producing agents. Typically, such compositions will be employed in combination with other therapies such as antibiotics in order to treat or cure infectious diseases. In many embodiments of the invention, anti-toxin compositions will be administered after symptoms of infection have become manifest. One particularly preferred embodiment of the invention comprises an anthrax anti-toxin composition.

### DESCRIPTION OF THE DRAWING

FIG. 1 presents a schematic representation of the anthrax life cycle.

### DEFINITIONS

Antibiotic: Antibiotics are compounds that suppress the growth of microorganisms, and include both compounds that result in cell death and compounds that result in stasis. Antibiotics used to treat anthrax infections include agents that inhibit bacterial cell wall synthesis (e.g., penicillin G, cephalosporins), agents that affect the function of the 30S or 50S ribosomal subunits (e.g., erythromycin, doxycyclin, chloramphenicol), and inhibitors of DNA replication, particularly via inhibition of DNA gyrase (e.g., ciprofloxacin).

Anti-toxin: An anti-toxin composition for use in accordance with the present invention is a composition that includes at least one ligand that binds to and interferes with one or more components of a microbial toxin. In certain preferred embodiments of the invention, the anti-toxin includes multiple toxin ligands, and in some cases includes at least one ligand to interact with each toxin component. Preferred anti-toxin compositions include anti-anthrax toxin ligands. Certain such anti-anthrax toxin compositions include one or more ligands that bind to and interfere with the anthrax protective antigen PA. Such PA ligands may, for example, interfere with PA oligomerization, so that an active pore is not formed in the presence of the ligand. Alternatively or additionally, PA ligands may block or clog the pore, or otherwise interfere with pore function. PA ligands are desirable components of an inventive anti-anthrax anti-toxin because they may exert a dominant negative effect at sub-stoichiometric ratios with PA. Inventive anti-anthrax anti-toxin compositions may alternatively or additionally include one or more ligands that binds to the anthrax edema factor (EF) or lethal factor (LF). Particularly preferred ligands for inclusion in inventive anti-toxin compositions are antibodies that specifically recognize the target molecule.

Isolated: The term “isolated” is used herein to refer to chemical entities (e.g., polypeptides, nucleic acids, lipids, carbohydrates, small molecules) that occur in nature but that are in a form other than that in which they occur in nature. In particular, “isolated” compounds are typically separated from at least one other compound with which they are associated when they occur in nature; in certain embodiments of the invention an isolated compound is partially pure or substantially pure. For example, an isolated compound may be 50%, 60%, 70%, 75%, 80%, 85%, 90%, 92%, 93%, 94%, 95%, 96%, 97%, 98%, 99%, or greater than 99% pure. An indication that a compound is isolated is not an indication that the particular compound was ever in the state in which that compound is found in nature. For example, a pure preparation of protein that is found in nature in human cells is considered to be an “isolated” protein even though the pure preparation was synthesized chemically, or was purified from a bacterial cell that had been engineered to express it, and was never expressed in a human cell.

Recombinant: A “recombinant” nucleic acid is one that has been engineered by the hand of man, particularly using the techniques of Recombinant DNA Technology (e.g., restriction, ligation, polymerase chain amplification, reverse transcription, transformation, etc.), and includes progeny of such molecules. For example, a vector including a cloned sequence is considered in the art to be a recombinant molecule even though the particular molecule in question was not itself subjected to restriction digestion and ligation.

Small molecule: “Small molecule is a term of art that is applied to organic compounds, typically having a molecular weight of less than 1500. Small molecules may be naturally-occurring compounds or chemically synthesized or prepared compounds.

### DESCRIPTION OF CERTAIN PREFERRED EMBODIMENTS OF THE INVENTION

The present invention provides compositions and methods for the treatment of microbial infections by the administration of anti-toxin. The invention is particularly applicable to infections of toxin-producing pathogenic bacteria. Particularly preferred embodiments of the invention provide treatments for infections with anthrax.

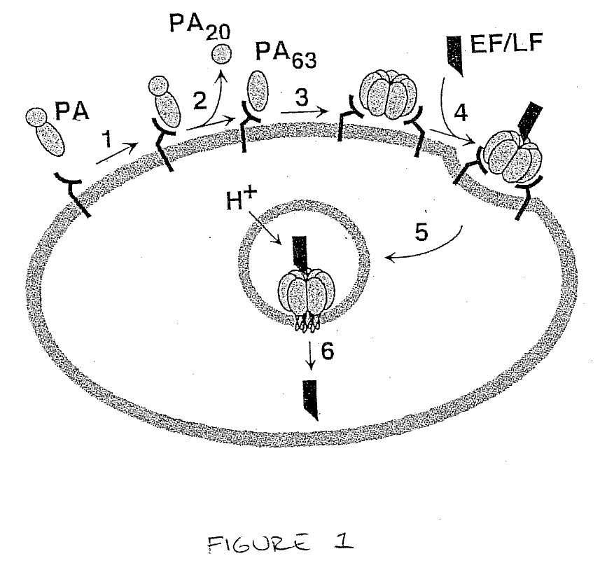

FIG. 1 depicts the infection cycle for the anthrax bacterium. As can be seen, the anthrax bacterium secretes three proteins, known as “protective antigen” (PA), “edema factor” (EF), and “lethal factor” (LF), that collectively make up the anthrax toxin.

Protective antigen is initially produced as an 83-kilodalton (kD) precursor protein (PA₈₃) that is bound by a receptor found on the surface of mammalian cells. The receptor-bound PA₈₃ is cleaved by furin-related proteases into a 20-kD fragment (PA₂₀) and a 63-kD fragment (PA₆₃) (Escuyer et al., Infect. Immunol. 59:3381, 1991; Klimpel et al., Proc. Natl. Acad Sci. USA 89:10277, 1992). PA₂₀ diffuses away, and PA₆₃ oligomerizes to form a pore structure comprised of seven PA₆₃ molecules Petosa et al., Nature 385:833, 1997; Milne et al., J. Biol. Chem. 269:20607, 1994). EF and LF bind to this pore structure. Oligomerization of the receptor-bound PA₆₃ molecules triggers receptor-mediated endocytosis of the pore structure, including any bound EF and/or LF, into an endosome. Acidification of the endosome triggers a conformational change in the pore structure that results in insertion of the pore structure in the endosomal membrane, forming an active pore that allows EF and/or LF to translocate into the cytosol (see, for example, Friedlander et al., J. Biol. Chem. 261:7123, 1986; Koehler et al., Mol. Microbiol. 5:1501, 1991).

Once delivered to the cytosol, LF and EF reek their havoc. EF is a calmodulin-dependent adenylate cyclase whose activity causes increases in intracellular cyclic AMP (CAMP) levels, and leads to the formation of ion-permeable pores in cell membranes, resulting in hemolysis. LF is a Zn²⁺-dependent protease that acts specifically in macrophages, where it cleaves various mitogen-activated protein (MAP) kinase kinases, eventually resulting in cell death. LF activity is thought to be primarily responsible for killing the host organism in fatal anthrax infections.

Anthrax anti-toxin compositions of the present invention preferably interfere with one or more of the activities of PA, EF, and/or LF.

Anti-toxin Compositions

In one aspect, the present invention provides anti-toxin compositions effective in the treatment of microbial infections. Inventive anti-toxin compositions include one or more binding components (i.e., ligands) that physically associate with microbial toxins and reduce or eliminate their ability to damage an infected host.

In preferred embodiments of the invention, the binding components present in the anti-toxin compositions are antibodies that bind specifically to toxin proteins. Such antibodies may be monoclonal or polyclonal, and may be of any class, although typically IgG antibodies are employed. In certain preferred versions of this embodiment of the invention, the antibodies are raised in an animal of the same species as the individual to whom the anti-toxin is to be administered. For example, antibodies raised in a human are preferably used for administration to a human. However, such species matching is not required. For example, antibodies raised in a non-human animal may be administered to human recipients. Non-human animals useful for raising antibodies to be administered to humans include, for example, mice, rats, cats, dogs, pigs, goats, sheep, cows, horses, or other animals that mount an immune response against the relevant toxin. Also, the invention encompasses antibody-containing compositions in which antibodies have been prepared other than in a human but have been “humanized” according to known techniques (e.g., raising antibodies in a mouse with a humanized immune system, or raising antibodies in a non-human animal and then substituting portions of the antibody chains with human chain sequences).

In certain preferred embodiments of the invention, a series of different antibody preparations are generated in different immunized host animals so that multiple different preparations are available for treatment of infected individuals (or individuals at risk of infection). For example, there is a risk that human subjects may be or may become allergic to one or more non-human antibody preparations. According to the present invention, it may be desirable to administer to a given human subject a first antibody preparation from a first immunized host for a period of time, and then to switch and administer a second antibody preparation from a second immunized host, etc. Switches may be made before or after the development of allergic or other undesirable symptoms associated with administration of a given preparation.

Alternative binding components for use in accordance with the present invention include non-antibody protein ligands, including so-called single-chain antibodies, which are single polypeptide chains that preserve the three-dimensional structure, and therefore the binding specificity, of an antibody molecule; small molecules that interact specifically with one or more toxin components; and any other compound that appropriately binds to and/or inactivates a microbial toxin.

Certain preferred anti-toxin compositions of the present invention contain multiple binding agents, each of which interacts with a different moiety within the relevant microbial toxin. For example, in one embodiment of the invention, the antitoxin comprises serum isolated from an individual (i.e., a human or a non-human animal) who has been immunized with a toxin preparation. Such serum will contain a collection of anti-bodies that recognize different epitopes in the toxin proteins with which the individual was immunized.

Antibodies or other ligands for use in accordance with the present invention may be prepared according to any available technique. For example, techniques for immunizing a non-human animal (e.g., a horse, mouse, rabbit, etc.) with a selected antigen are well known. According to the present invention, the immunizing antigen would be a bacterial toxin or toxin component, preferably an anthrax toxin. Typically, it will be preferably to use all toxin proteins (e.g., PA, EF, and LF together) as an immunogen. However, individual proteins, or portions of proteins (e.g., only PA₆₃, or only peptides corresponding to known immunodominant eptiopes in PA, EF, and/or LF, etc.) may be employed. Immunogen compositions may represent crude preparations (e.g., from natural sources), but preferably are pure (e.g., are purified polypeptides, for example prepared according to techniques of Recombinant DNA Technology, or chemically synthesized).

Anti-toxin compositions of the present invention are preferably formulated according to established principles of pharmacology for administration to humans or nonhuman mammals (e.g., horses, cows, goats, sheep, dogs, cats, or other domesticated animals susceptible to the relevant microbial infection). In certain preferred embodiments, inventive anti-toxin compositions include or are admininstered together with other therapies useful in treating the relevant microbial infection. For example, inventive anti-toxin compositions may include or be administered together with one or more antibiotics.

Different antibiotic regimens are recommended for treating different bacterial infections. For example, recommended therapies for anthrax infection include penicillins (e.g., Penicillin G), erythromycin, doxycycline, chloramphenicol, ciprofloxacin, cephalexin, cefazolin, and/or cefadroxil. Such compounds may be included with antitoxins of the present invention. Inventive therapies may also be combined with pH-modifying agents as described, for example, in U.S. patent application serial No. 60/338,618, entitled “Methods for Preventing or Treating Disease Mediated by Toxin-Secreting Bacteria”, filed Nov. 13, 2001 and incorporated herein by reference.

Whatever the active components (e.g., only anti-toxin ligands, or anti-toxin ligand plus antibiotic(s), pH-modifying compounds, etc.) of the inventive pharmaceutical composition, it is formulated according to known techniques, typically for delivery by injection.

One or more of the compounds included in inventive pharmaceutical compositions may be presented in the form of a pharmaceutically acceptable derivative such as a salt, ester, salts of an ester, or any other adduct or derivative which upon administration to a patient in need is capable of providing, directly or indirectly, a compound as otherwise described herein, or a metabolite or residue thereof (e.g., a prodrug). As used herein, the term “pharmaceutically acceptable salt” refers to those salts which are, within the scope of sound medical judgement, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describe pharmaceutically acceptable salts in detail (see, for example, J. Pharmaceutical Sciences, 66:1, 1977, incorporated herein by reference). The salts can be prepared in situ during the final isolation and purification of the compounds of the invention, or separately by reacting the free base function with a suitable organic acid.

Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hernisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

As used herein, the term “pharmaceutically acceptable ester” includes esters that hydrolyze in vivo and those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butyrates, acrylates and ethylsuccinates.

Furthermore, the term “pharmaceutically acceptable prodrugs” as used herein refers to those prodrugs that are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the invention. Generally, the term “prodrug” refers to compounds that are rapidly transformed in vivo to yield the parent compound of the above formula, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

Inventive pharmaceutical compositions may comprise a pharmaceutically acceptable carrier, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Except insofar as any conventional carrier medium is incompatible with the anti-toxin compounds of the invention, such as by producing any undesirable biological effect or otherwise interacting in a deleterious manner with any other component(s) of the pharmaceutical composition, its use is contemplated to be within the scope of this invention.

Some examples of materials that can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Treating Infections

Inventive anti-toxin compositions block or reduce the poisoning effects of microbial toxins and can usefully be employed at any time during a microbial infection either prior to production of toxin components, or so long as any such toxin components remain in the infected individual. As such, inventive compositions provide a useful adjunct to antibiotic therapy. Moreover, inventive compositions may usefully be employed even after the development of symptoms of infection. Inventive compositions are therefore particularly useful for the treatment of anthrax infections, for which there is no effective treatment that can be initiated after the development of clear symptoms.

Symptoms of anthrax infection usually appear within 7-10 days of exposure, and vary depending on the source and location of the infection. There are three classifications of anthrax infection: cutaneous, intestinal, and inhalation. Historically, most anthrax infections have been cutaneous infections that occur when the bacteria enter a cut or abrasion on the skin, for example when a farmer or woolhandler in handling contaminated wool, hides, leather or hair products (especially goat hair) of infected animals. Skin infection begins as a raised itchy bump that resembles an insect bite but within 1-2 days develops into a vesicle and then a painless ulcer, usually 1-3 cm in diameter, with a characteristic black necrotic (dying) area in the center. Lymph glands in the adjacent area may swell. About 20% of untreated cases of cutaneous anthrax result in death.

The intestinal disease form of anthrax may follow the consumption of contaminated meat and is characterized by an acute inflammation of the intestinal tract. Initial signs of nausea, loss of appetite, vomiting, fever are followed by abdominal pain, vomiting of blood, and severe diarrhea. Intestinal anthrax results in death in 25% to 60% of cases.

Inhalation anthrax poses the most significant diagnosis challenge, both because the initial symptoms resemble those of a common cold and because, if untreated, inhalation anthrax is almost always fatal. Although antibiotics can be effective, they will not help once the infection has progressed beyond a certain point. Unfortunately, once symptoms progress beyond the very early stage, cold-like symptoms, it is often too late for antibiotics alone. At this point, symptoms may progress to severe breathing problems and shock, eventually resulting in death.

One advantage of the inventive anti-toxin compositions for use in the treatment of anthrax infections is that they may be utilized at a relatively late stage of infection. For example, inventive compositions may be administered after the development of symptoms of inhalation or ingestion anthrax, and possibly even after the developm,ent of late-stage symptoms such as breathing problems, etc.

Inventive anti-toxin therapy may be employed alone to treat microbial infections, in which case the immune system of the infected host will be responsible for clearing the infection, or alternatively (and generally preferably) may be employed in combination with one or more other therapies including, for example, antimicrobial therapies or pH-modulating therapies. In general, lower doses of anti-toxin compositions are expected to be necessary when anti-toxin therapy is administered in combination with antibiotic or other therapy.

Inventive anti-toxin compositions that include a ligand that binds to and/or interferes with the formation or activity of a multi-subunit toxin component are generally administered at lower doses than other compositions. Typically, it is preferable to utilize a ligand that need only be present in one or a few doses per multi-subunit entity in order to exert its effect. Such ligands can exert potent effects even at sub-stoichiometric levels as compared with their binding partner.

Those of ordinary skill in the art will understand that it is not necessary to administer sufficient quantity of anti-toxin composition to bind to and/or interfere with every toxin molecule in an infected individual. Rather, it is sufficient to provide a sufficient amount of anti-toxin composition that the worst effects of the toxin in the infected individual are reduced or eliminated. In particular, it is desirable that a sufficient amount of anti-toxin composition be administered to prevent death. It is noted that, without treatment, inhalation anthrax infections are almost invariably fatal.

Inventive pharmaceutical compositions for the treatment of microbial infections can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid dosage forms for oral administration include capsules, tablets, pills, powders, and granules. In such solid dosage forms, inventive anti-microbial agents are preferably mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar—agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Inventive pharmaceutical compositions can also be administered in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Injectable preparations include, for example, sterile injectable aqueous or oleaginous suspensions and may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Inventive injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of an inventive anti-toxin, it may be desirable to slow the absorption of the drug from a subcutaneous or intramuscular injection. This may be accomplished, for example, by the use of a liquid suspension of crystalline or amorphous material with poor water solubility. The rate of absorption of the drug then depends upon its rate of dissolution which, in turn, may depend upon crystal size and crystalline form.

Alternatively, delayed absorption of a parenterally administered anti-toxin preparation may be accomplished by dissolving or suspending the anti-toxin in an oil vehicle. Injectable depot forms are made by forming microencapsule matrices of the drug in biodegradable polymers such as polylactide-polyglycolide. Depending upon the ratio of anti-toxin to polymer and the nature of the particular polymer employed, the rate of drug release can be controlled. Examples of other biodegradable polymers include poly(orthoesters) and poly(anhydrides). Depot injectable formulations can also be prepared by entrapping an anti-toxin in liposomes or microemulsions that are compatible with body tissues.

In some instances, it may be desirable to formulate inventive anti-toxin-containing composition for rectal or vaginal administration. For example, suppositories may be prepared, preferably mixing inventive anti-toxins with suitable non-irritating excipients or carriers such as cocoa butter, polyethylene glycol or a suppository wax which are solid at ambient temperature but liquid at body temperature and therefore melt in the rectum or vaginal cavity and release the active compound.

It may be desirable to administer topical or transdermal formulations of inventive compositions, for example for the treatment of cutaneous anthrax infections. Dosage forms for such topical or transdermal administration include ointments, pastes, creams, lotions, gels, powders, solutions, sprays, inhalants or patches. The active component (i.e., the anti-toxin, optionally in combination with one or more other anti-microbial agents, is admixed under sterile conditions with a pharmaceutically acceptable carrier and any needed preservatives or buffers as may be required.

Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the inventive anti-toxin in the proper medium. Absorption enhancers can also be used to increase the flux of the compound across the skin. The rate can be controlled by either providing a rate controlling membrane or by dispersing the compound in a polymer matrix or gel.

Identification of Useful Anti-Toxin Ligands

As noted herein, preferred ligands for use in inventive anti-toxin compositions are antibodies. However, other chemical compounds may be employed. In general, useful ligands may be identified by analysis of test agents in one or more model systems that mimics one or more aspects of the relevant microbial infection.

Both in vitro and in vivo models for anthrax infection are available. For example, models of PA₆₃ heptamerization (see, for example, Sellman et al., Science 292:695, 2001, incorporated herein by reference), binding of LF to PA₆₃ on CHO cells (Miller et al., Biochemistry 38:10432, 1999, incorporated herein by reference), and adenylate cyclase activity exist, as do n vitro (Milne et al., Mol. Microbiol. 15:661, 1995, incorporated herein by reference) and in vivo models of anthrax infection (see, for example, Ivins et al., Appl. Environ. Microbiol/55:2098, 1989, incorporated herein by reference). Test agents may be characterized in one or more such assays in order to identify those that may usefully be employed as toxin component ligands in accordance with the present invention.

I claim:

1. A method of preventing or treating disease mediated by Bacillus anthracis in an individual, comprising administering to the individual an effective amount of chloroquine.

2. The method of claim 1, further comprising administering to the individual an effective amount of an antibiotic.

3. The method of claim 2, wherein the antibiotic is selected from the group consisting of penicillin G, erythromycin, doxycycline, chloramphenicol, ciprofloxacin, cephalexin, cefazolin, and cefadroxil.

4. The method of claim 1, wherein the effective amount of chloroquine is between about 0.001 mg/kg to about 40 mg/kg of body weight.

5. The method of claim 1, wherein the effective amount of chloroquine is between about 0.01 mg/kg to about 40 mg/kg of body weight.

6. The method of claim 1, wherein the effective amount of chloroquine is between about 0.001 mg/kg to about 25 mg/kg of body weight.

7. The method of claim 1, wherein the effective amount of chloroquine is between about 0.01 mg/kg to about 25 mg/kg of body weight.

8. The method of claim 1, wherein the effective amount of chloroquine is between about 0.001 mg/kg to about 10 mg/kg of body weight.

9. The method of claim 1, wherein the effective amount of chloroquine is between about 0.01 mg/kg to about 10 mg/kg of body weight.

10. The method of claim 1, wherein the effective amount of chloroquine is between about 0.001 mg/kg to about 1.0 mg/kg of body weight.

11. The method of claim 1, wherein the effective amount of chloroquine is between about 0.01 mg/kg to about 1.0 mg/kg of body weight.

12. The method of claim 1, wherein the effective amount of chloroquine is about 25 mg/kg or greater of body weight.

13. The method of claim 1, wherein the effective amount of chloroquine is between about 25 mg/kg to about 40 mg/kg of body weight.

14. The method of claim 2, wherein the effective amount of the antibiotic is between about 1 mg/kg to about 20 mg/kg of body weight.

15. The method of claim 1, further comprising administering to the individual an effective amount of an anti-toxin.

16. The method of claim 1, wherein the step of administering is performed after symptoms of infection by Bacillus anthracis have become manifest.

17. A pharmaceutical composition comprising:
a pharmaceutically suitable carrier;
chloroquine; and
an antibiotic.

18. The pharmaceutical composition of claim 17, further comprising an anti-toxin.

19. The pharmaceutical composition of claim 17, wherein the antibiotic is selected from the group consisting of penicillin G, erythromycin, doxycycline, chloramphenicol, ciprofloxacin, cephalexin, cefazolin, and cefadroxil.
